(19) United States
(12) Patent Application Publication
(10) Pub. No.: US 2017/0340825 A1
(43) Pub. Date: Nov. 30, 2017

(54) COMPONENT FOR A SYRINGE AND SYRINGE

(71) Applicant: Gerresheimer Regensburg GmbH, Regensburg (DE)

(72) Inventors: Juergen PFRANG, Kallmuenz (DE); Bernhard SCHMITT, Wackersdorf (DE)

(73) Assignee: Gerresheimer Regensburg GmbH, Regensburg (DE)

(21) Appl. No.: 15/600,836

(22) Filed: May 22, 2017

(30) Foreign Application Priority Data
May 24, 2016 (DE) 10 2016 109 505.2

(51) Int. Cl. A61M 5/315; A61M 5/31

(52) U.S. Cl. CPC A61M 5/31505; A61M 5/31576; A61M 5/315; A61M 5/31511; A61M 2005/3117

(57) ABSTRACT

A component for a syringe is disclosed. The component has formed a structure which has a friction-reducing effect due to its shape in the syringe. The component can be, for example, a cylinder, a piston or a seal for the syringe. The structure comprises, for example, elevations or depressions which have a linear, punctiform, elliptical or polygonal design. Also disclosed is a syringe which has at least one such component. The use of a component with a friction-reducing structure makes it unnecessary to use an additional friction-reducing coating, for example silicone, which can lead to undesirable effects on the medicament to be administered with the syringe.

This nonprovisional application claims priority under 35 U.S.C. §119(a) to German Patent Application No. 10 2016 109 505.2, which was filed in Germany on May 24, 2016, and which is herein incorporated by reference.

### BACKGROUND OF THE INVENTION

### Field of the Invention

The invention relates to a component for a syringe and a syringe. In particular, the invention relates to a component for a syringe which is constructed to reduce friction.

### Description of the Background Art

Syringes are used predominantly in the medical field and, as is known, comprise a cylinder in which a piston is movably mounted. Furthermore, a seal can be provided between the cylinder and piston. The seal can also form an end of the piston, for example in the form of a plug of rubber or other elastic material. By moving the piston in the cylinder, an active ingredient can be administered, for example to a patient, or a sample, for example a blood sample, can be collected from a patient, in each case via a cannula attached to the syringe. In this case, it is desirable that the movement of the piston in the cylinder be as low as possible with low friction. Excessive friction can lead to jerky movements of the piston during force exertion on the piston. Such a jerky movement prevents accurate dosage when administering an active ingredient preparation as well as during sampling and results in additional stress on the patient since a jerky movement of the piston often also results in additional movements of the cannula in the patient's body.

To reduce the friction between piston and cylinder, it is known to provide a layer with friction-reducing properties, such as a silicone layer or a PTFE layer, between piston and cylinder.

European patent application EP 2 653 484 A1, which corresponds to U.S. Pat. No. 9,394,391, describes the chemical modification of a surface for the purpose of friction reduction, among other things with regard to syringes. For this purpose a polymer layer is produced on the surface of a monomer. A similar process is described in the European patent EP 2 752 436 B1, which corresponds to U.S. Pat. No. 9,540,493.

However, a layer of a material such as silicone can lead to pharmaceutically undesirable interactions between the material of the layer and some active ingredients, for example biosimilars. Some materials, such as PTFE, exhibit a desirable friction-reducing effect, but are at the same time relatively hard, so that the seal suffers therefrom, that means in this case, the piston does not close off sufficiently tightly with the cylinder.

### SUMMARY OF THE INVENTION

It is therefore an object of the invention to provide a component for a syringe so that, on the one hand, the cylinder closes sufficiently tightly with the piston or the seal, and at the same time the friction between the cylinder and the piston, or between the cylinder and the seal, compared to prior art, is reduced without the result of pharmaceutically undesirable interactions.

In an exemplary embodiment, a component for a syringe has a structure which is formed by shaping a region of the component. The structure is designed to have a friction-reducing effect by virtue of its shape in the syringe. This means that the form is such that when the component is inserted into the syringe in a known manner known per se, the component in the syringe can be moved with a friction against at least one other component which friction is reduced, compared to the use of a component without said structure.

An advantage of a component according to the invention is that a desired reduction in friction can be achieved without introducing an additional material into the syringe. In this way, pharmaceutically undesirable effects of an additional material on an active substance to be administered or a sample to be taken are reliably excluded.

In exemplary embodiments, the component for a syringe can be, for example, a cylinder for the syringe, or a piston for the syringe, or a seal for a piston of the syringe.

If the component is a cylinder for a syringe, the structure can be located on an inner side of a casing of the cylinder. In this way, the friction on a piston or on a seal for the piston of the syringe can be reduced by the structure.

If the component is a piston for a syringe, the structure can be arranged on the piston such that it faces a casing of a cylinder for the syringe in the syringe, that is to say when the piston is inserted into the syringe. In this way, the friction on a cylinder for the syringe can be reduced by the structure.

If the component is a seal for a piston of a syringe, the structure can be arranged on the seal such that it opposes a casing of a cylinder for the syringe in the syringe, i.e. when the piston carrying the seal is introduced into the syringe. In this way, the friction on a cylinder for the syringe can be reduced by the structure.

In an exemplary embodiment, the structure comprises a plurality of structural elements. The structural elements can be homogeneous or different.

In particular, in an exemplary embodiment, each such structural element present on the component is of one of the following types, independently of the remaining structural elements present on the component: linear depression, linear elevation, punctiform depression, punctiform elevation, depression with elliptical design, elevation with elliptical design, depression with polygonal design, elevation with polygonal design. In this context, elliptical especially includes the special case circular.

The structural elements can be regularly arranged on the component according to the invention. The structural elements can also be arranged irregularly on the component according to the invention.

In an exemplary embodiment, the structural elements can be arranged on the component according to the invention such that a spacing between adjacent structural elements is in the range of 10 μm to 1 mm. In this case, in exemplary embodiments the structural elements can be arranged regularly, and the spacing between adjacent structural elements can have a fixed value from the range. It is likewise possible, for example, that the arrangement of the structural elements is irregular, and the spacing between adjacent structural elements is not constant, but assumes various values from the range mentioned, depending on the pair of adjacent structural elements.

In some embodiments, a width of a structural element can be in the range from 10 μm to 100 μm. In the same way as in the case of the spacing, in an exemplary embodiment of this case, all the structural elements can have the same value from the range for the width, or the structural elements can have different values for the width which are in the range.

If the structural element is an elevation, a height of the structural element in embodiments can be in the range 1 μm to 100 μm. If the structural element is a depression, a depth of the structural element in embodiments can be in the range 1 μm to 100 μm. In the same way as in the case of the spacing, in an exemplary embodiment of this case, all the structural elements can have the same value from the range for the height or depth, or the structural elements can have different values for the height or depth which are in the said region.

The structural elements can be arranged in certain patterns on the component. In particular, the structural elements can be arranged on the component such that they are parallel or perpendicular to the direction of movement of the piston in the cylinder of the syringe, or at an angle of between 30 degrees and 60 degrees.

The structure can comprise at least two groups of structural elements on the component. The structural elements within a group are homogeneous. The structural elements of two groups differ with respect to at least one of the following features discussed above: shape, width, height, depth, orientation with respect to a direction of movement of the piston in the cylinder of the syringe.

In an embodiment, a syringe comprises at least one component according to the invention of the type described above. In this way, the friction between components of the syringe, i.e. between the cylinder and the piston or between the cylinder and the seal for the piston, is reduced.

In an exemplary embodiment, not only two components of the syringe according to the invention are of the type described above, but the respective structures present on the component according to the invention can be such that an additional friction-reducing effect results from the interaction of the respective structures of the components.

Syringes are generally made of plastic. The structures as described can be produced during the production of the respective component of the syringe, for example by suitable molds by an injection molding process or by pressing with a correspondingly shaped molding tool, without the invention being limited to components produced by the aforementioned production methods for syringes.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIGS. 1A and 1B are schematic embodiments of syringes in which the present invention can be used advantageously;

FIGS. 2 to 4 are exemplary embodiments of components for a syringe according to the invention provided with a structure;

FIGS. 5 to 8 are exemplary embodiments of a structure, such as can be formed on a component according to the invention, in plan view;

FIGS. 9 to 12 are exemplary embodiments of a structure, such as can be formed on a component according to the invention, in plan view;

FIGS. 13 to 15 are exemplary embodiments of a structure, such as can be formed on a component according to the invention, in plan view;

FIGS. 16 to 19 are sectional views through various structural elements of a structure as can be formed on a component according to the invention;

FIGS. 20 and 21 show a plan view of examples of a combination of structures as can be formed on a component according to the invention; and

FIG. 22 is a plan view of another example of a combination of structures as can be formed on a component according to the invention.

### DETAILED DESCRIPTION

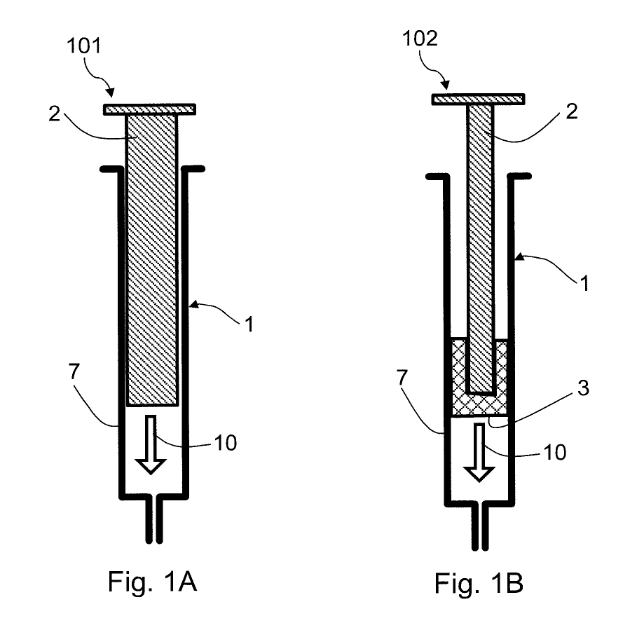

FIGS. 1A and 1B schematically show the construction of two syringes 101 and 102 known per se. In FIG. 1A, the syringe 101 comprises a cylinder 1 in which a piston 2 is movably arranged. The cylinder 1 has, in particular, a casing 7. In FIG. 1B, the syringe 102 comprises a cylinder 1, in which a piston 2 is arranged movably. A seal 3 is attached to the piston 2 to seal the piston 2 against the cylinder 1. Cylinder 1, piston 2, and seal 3 form the components of the syringe 101 or 102 in the sense of this application. For the administration of an active substance or for the removal of a sample, the piston 2 is moved in a manner known per se relative to the cylinder 1, along the direction indicated by the arrow 10 or opposite thereto. In the case of the syringe 101 between the piston 2 and the cylinder 1, and in the case of the syringe 102 between the seal 3 and the cylinder 1, friction forces act. These frictional forces can, for example, complicate the administration of an active substance or the removal of a sample, as already mentioned above. In the case of components 1, 2, 3 of the syringe 101 or 102 according to the invention, these frictional forces are reduced without having to introduce an additional, pharmaceutically undesirable, material into the syringe 101, 102.

FIGS. 2 to 4 show three exemplary embodiments of how components according to the invention of a syringe can be designed. A component according to the invention has a region 4 with a structure 5. In the exemplary embodiments shown, each structure 5 comprises a plurality of structural elements 6. The piston 2 and the cylinder 1 are each shown only partially.

In the exemplary embodiment of FIG. 2, a region 4 of the cylinder 1 is formed as a structure 5. The friction between the piston 2 and the cylinder 1 is thereby reduced. The structure 5 is formed on an inner side 8 of a casing 7 of the cylinder 1. Here, a piston 2 without a seal is shown. Likewise, a cylinder 1, as shown in FIG. 2, could also be used with a seal 3 for the piston 2 in the case of a syringe 102 (see FIG. 1B).

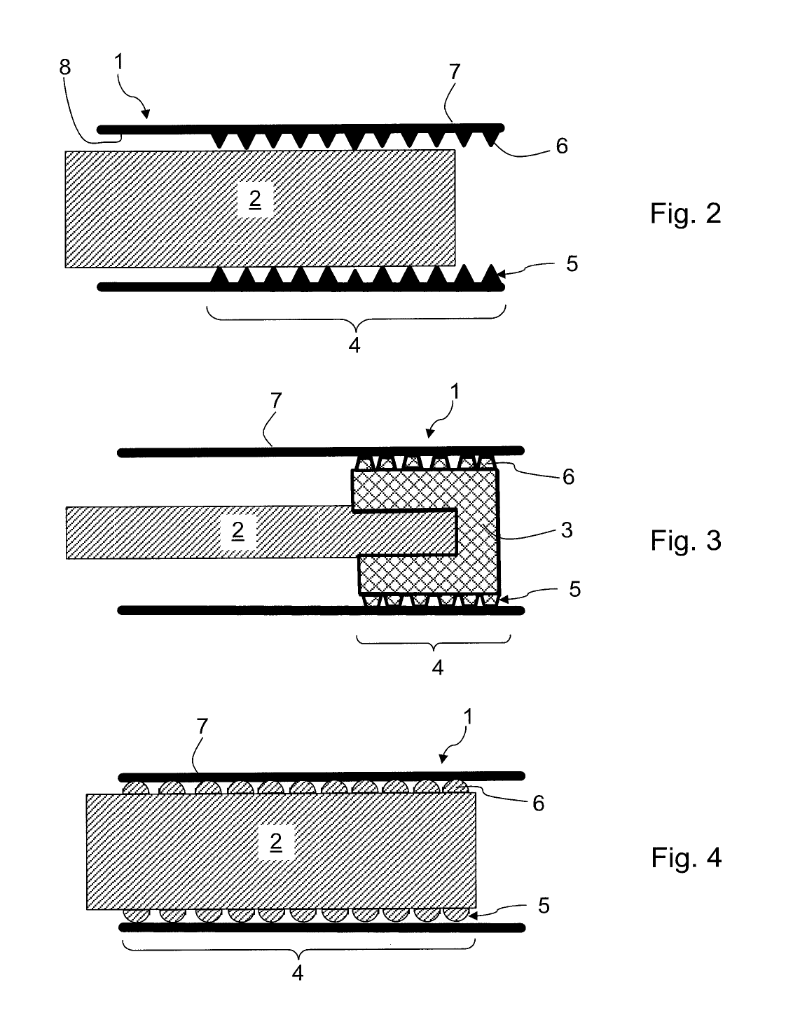

In the exemplary embodiment of FIG. 3, a region 4 of a seal 3 for the piston 2 is formed as a structure 5. The friction between the seal 3 and the cylinder 1 is thereby reduced. The piston 2 with the seal 3 is shown inserted into a cylinder 1 of a syringe 102 (see FIG. 1B). The region 4 of the seal 3, which is formed as a structure 5, is arranged opposite a casing 7 of the cylinder 1.

In the exemplary embodiment of FIG. 4, a region 4 of the piston 2 is formed as a structure 5. The friction between the piston 2 and the cylinder 1 is thereby reduced. The piston 2 is shown here inserted into a cylinder 1 of a syringe 101 (see FIG. 1B). The region 4 of the piston 2, which is formed as a structure 5, is arranged opposite a casing 7 of the cylinder 1.

In all cases, it is a region 4 of the respective component according to the invention, i.e. cylinder 1 in the case of FIG. 2, seal 3 in the case of FIG. 3, and piston 2 in the case of FIG. 4, which is formed as the structure 5. A further material is thus not introduced into the syringe.

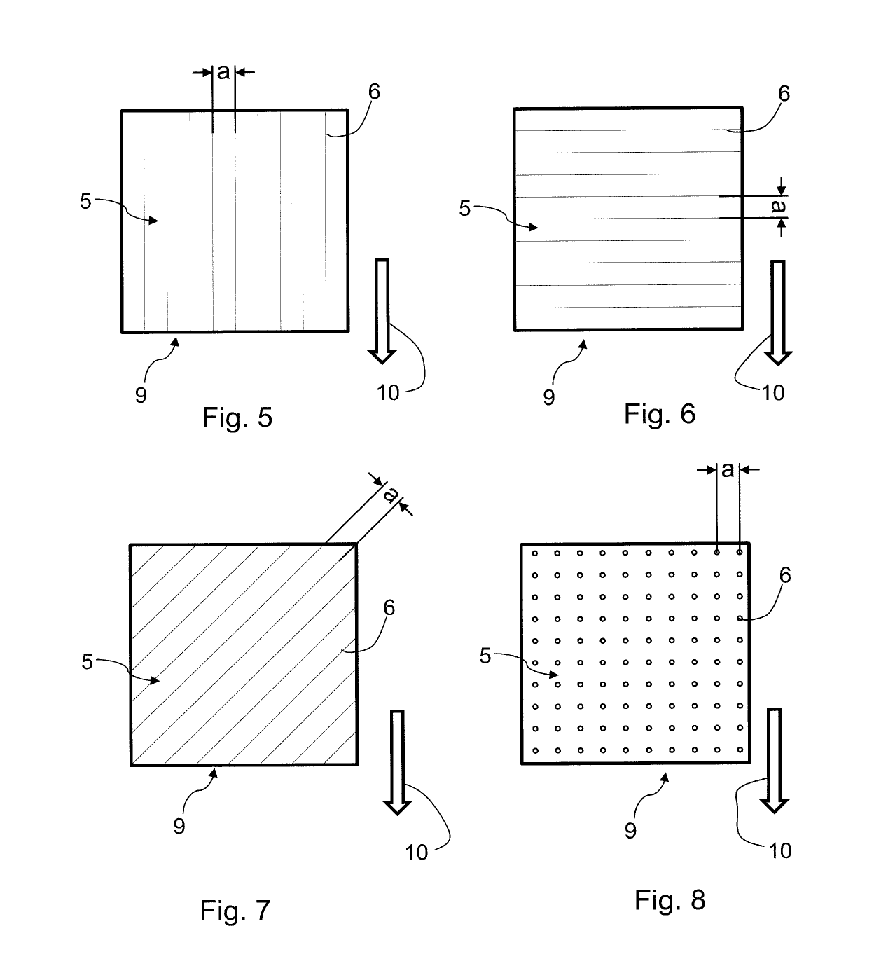

FIGS. 5 to 8 show, in plan view, exemplary embodiments of a structure 5, as can be formed on a component 1, 2, 3 according to the invention. Each structure 5 comprises a plurality of structural elements 6. Here, in each case, only a section 9 of a component is shown, whereby it is irrelevant whether the component is a cylinder 1, a piston 2 or a seal 3. The arrow indicates the direction 10 already shown in FIGS. 1A and 1B.

In the exemplary embodiments of FIGS. 5, 6 and 7, the structural elements 6 are linear-shaped; they can be linear-shaped elevations or linear-shaped depressions. In the exemplary embodiment of FIG. 8, the structural elements 6 are punctiform; they can be punctiform elevations or punctiform depressions. In the exemplary embodiments shown in FIGS. 5 to 8, the arrangement of the structural elements 6 is regular. The arrangement of the structural elements 6 is characterized, in particular, by a spacing a between adjacent structural elements 6. As can be seen from the figures, in the case of FIG. 5, the linear-shaped structural elements 6 are aligned parallel to the direction 10, in the case of FIG. 6, perpendicular thereto. In the exemplary embodiment of FIG. 7, the linear-shaped structural elements 6 form an angle of 45 degrees with the direction 10.

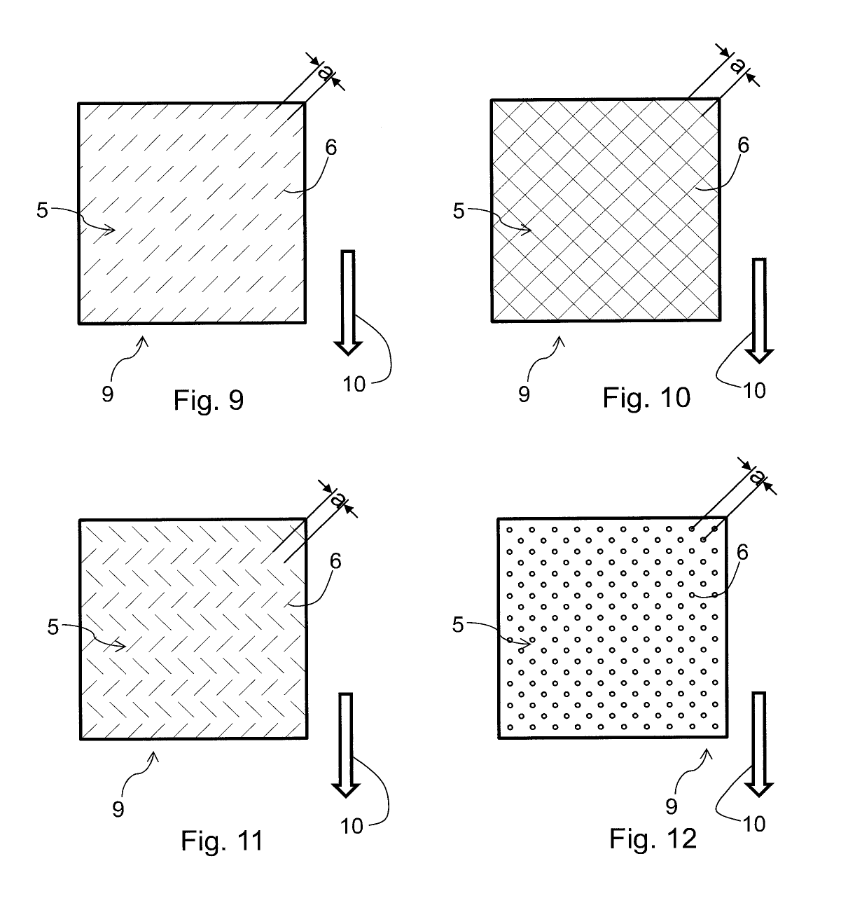

FIGS. 9 to 12 show, in plan view, further exemplary embodiments of a structure 5, as can be formed on a component 1, 2, 3 according to the invention. Each structure 5 comprises a plurality of structural elements 6. Here, in each case, only a section 9 of a component 1, 2, 3 is shown, whereby it is irrelevant whether the component is a cylinder 1, a piston 2 or a seal 3. The arrow indicates the direction 10 already shown in FIGS. 1A and 1B. In the exemplary embodiments shown in FIGS. 9 to 12, the structural elements 6 are arranged regularly, the structures 5 are each characterized by a spacing a between adjacent structural elements 6. In the exemplary embodiment of FIG. 9, the structural elements 6 are linear-shaped, for example, linear elevations or depressions, but in contrast to FIG. 7, no continuous lines. The structural elements include an angle in the range of 30 degrees to 60 degrees with the direction 10. In the exemplary embodiment of FIG. 10, the structural elements 6 form two groups of linear-shaped structural elements which intersect at a right angle and enclose an angle of 45 degrees with the direction 10. In the exemplary embodiment of FIG. 11, the structural elements 6 form two groups of linear-shaped structural elements which are arranged at a right angle to one another and enclose an angle of 45 degrees with the direction 10. FIG. 12 shows a further regular arrangement of punctiform structural elements 6.

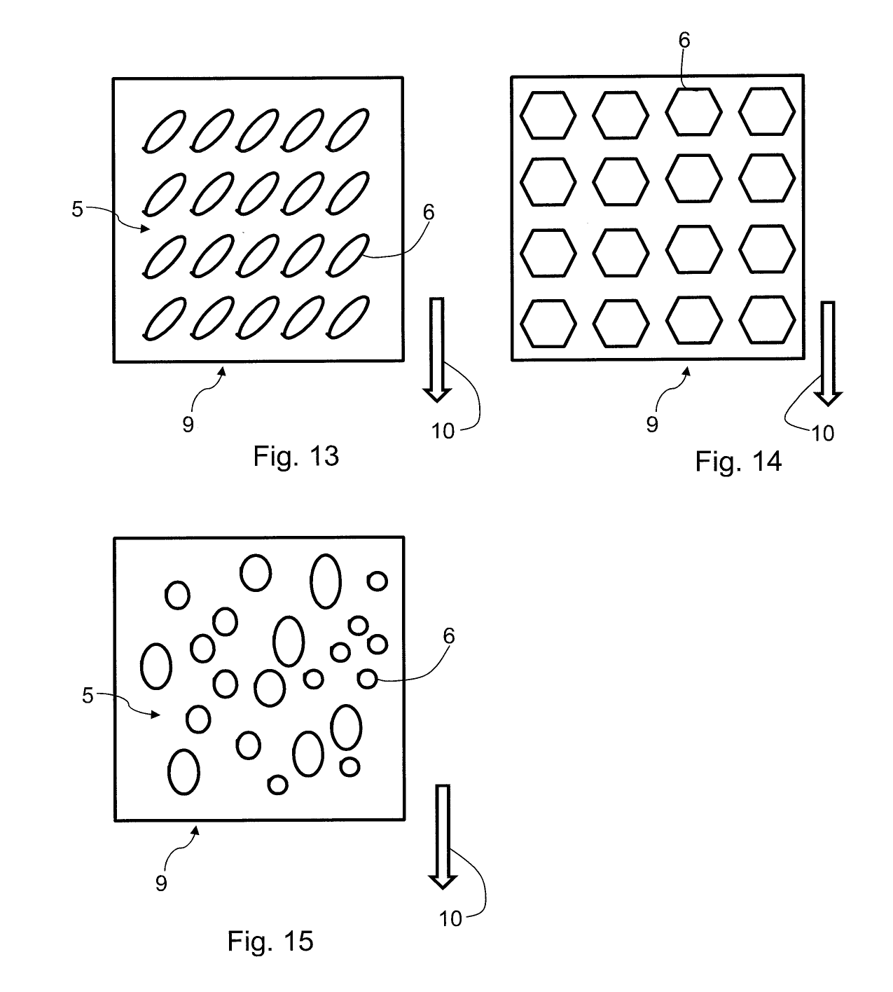

FIGS. 13 to 15 show, in plan view, further exemplary embodiments of a structure 5, as can be formed on a component 1, 2, 3 according to the invention. Each structure is composed of a plurality of structural elements 6. Here, in each case, only a section 9 of a component 1, 2, 3 is shown, whereby it is irrelevant whether the component is a cylinder 1, a piston 2 or a seal 3. The arrow indicates the direction 10 already shown in FIGS. 1A and 1B.

FIG. 13 shows regularly arranged structural elements 6 of elliptical cross section. FIG. 14 shows regularly arranged structural elements 6 of polygonal, in this case in particular hexagonal, cross section. FIG. 15 shows irregularly arranged structural elements 6, which also differ in size and shape.

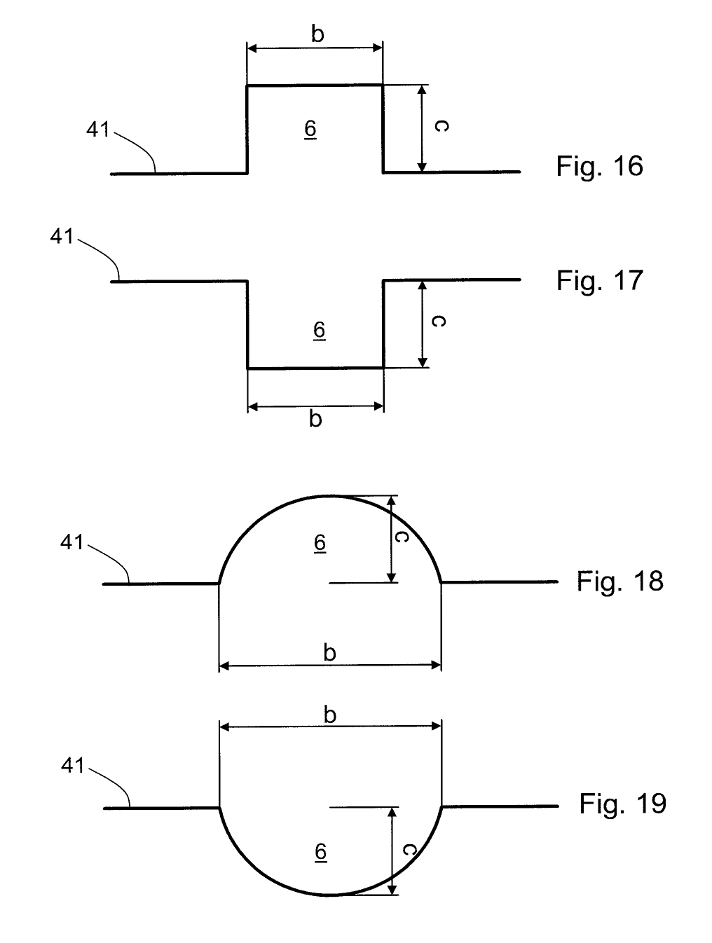

FIGS. 16 to 19 show exemplary embodiments of sectional views through structural elements 6, as can occur in structures on components 1, 2, 3 according to the invention. The structural elements 6 are characterized by a width b. In the exemplary embodiments of FIGS. 16 and 18, the structural elements 6 form elevations with respect to a reference level 41 of the component 1, 2, 3, and are characterized by a height c relative to this reference level 41. In the exemplary embodiments of FIGS. 17 and 19, the structural elements 6 form depressions opposite a reference level 41 of the component 1, 2, 3, and are characterized by a depth c relative to this reference level 41. The reference level 41 is, for example, given by a surface of the respective component 1, 2, 3 in the region between the structural elements 6. The exemplary embodiments of FIGS. 16 and 17 show sectional views through linear-shaped structural elements 6 with a rectangular cross-section; the exemplary embodiments of FIGS. 18 and 19 show sectional views through structural elements 6 with a circular-shaped ground plot and a rounded cross-section.

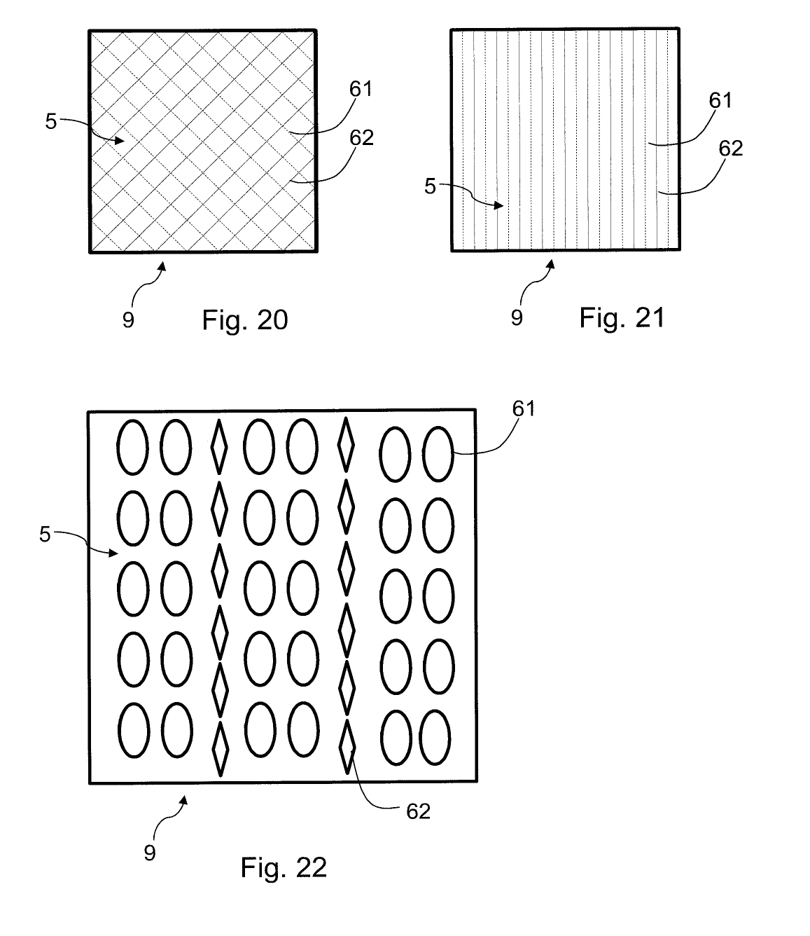

FIG. 20 and FIG. 21 show, in plan view, an example of a component 1, 2, 3 according to the invention. Only a section 9 of the component 1, 2, 3 is shown, respectively. Here, the structure 5 comprises two groups of structural elements, a group of first structural elements 61 and a group of second structural elements 62. The first structural elements 61 are homogeneous among one another. The second structural elements 62 are homogeneous to one another. The first structural elements 61 differ from the second structural elements 62. For example, the first structural elements 61 can have the form of broken lines while the second structural elements 62 have the form of solid lines. In FIG. 20, the first structural elements 61 and the second structural elements 62 are arranged such that the structural elements penetrate each other mutually. In FIG. 21, the first structural elements 61 are arranged parallel to the second structural elements 62. During use, an arrangement as in FIG. 21 favors a hooking, so that an arrangement according to FIG. 20 is preferred.

FIG. 22 shows a plan view of a further example of a component 1, 2, 3 according to the invention. Only a section 9 of the component 1, 2, 3 is shown. As in the examples of FIGS. 20 and 21, the structure 5 comprises two groups of structural elements, a group of first structural elements 61 and a group of second structural elements 62. The first structural elements 61 are homogeneous among one another. The second structural elements 62 are homogeneous to one another. The first structural elements 61 have an elliptical ground plot, the second structural elements 62 have a polygonal, in this case a rhombic, ground plot.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A component for a syringe, the component comprising:
a structure in which a region of the component is formed,
wherein the structure is constructed to reduce friction by its shape in the syringe.

2. The component as claimed in claim 1, wherein said component is a cylinder for a syringe, and said structure is located on an inner side of a casing of the cylinder.

3. The component as claimed in claim 1, wherein said component is a piston for a syringe, and wherein said structure is arranged on said piston so that said structure is located opposite a casing of the cylinder for the syringe when said structure of the piston is inserted into the syringe.

4. The component as claimed in claim 1, wherein the component is a seal for a piston of the syringe, and wherein the structure is arranged on the seal such that it is arranged in the syringe against a casing of a cylinder for the syringe.

5. The component as claimed in claim 1, wherein the structure comprises a plurality of structural elements, and wherein the structural elements are homogeneous or different.

6. The component as claimed in claim 5, wherein at least one of the plurality of structural elements is a: linear depression, linear elevation, punctiform depression, punctiform elevation, depression with elliptical design, elevation with elliptical design, depression with polygonal design, or elevation with polygonal design.

7. The component as claimed in claim 5, wherein the plurality of structural elements are arranged regularly or irregularly.

8. The component as claimed in claim 5, wherein a spacing between structural elements is in a range of 10 μm to 1 mm.

9. The component as claimed in claim 5, wherein a width of a structural element is in a range of 10 μm to 100 μm.

10. The component as claimed in claim 5, wherein a height or a depth of a structural element is in a range 1 μm to 100 μm.

11. The component as claimed in claim 5, wherein the structural elements are arranged on the component such that, with respect to a direction of movement of the piston in the cylinder of the syringe, the structural elements are substantially parallel or substantially perpendicular or at an angle between 30 degrees and 60 degrees.

12. The component as claimed in claim 5, wherein the structure comprises at least two groups of structural elements, wherein the structural elements are homogeneous within a group, and wherein the structural elements of two groups differ with respect to at least one of the following features: shape, width, height, depth, or orientation with respect to a direction of movement of the piston in the cylinder of the syringe.

13. A syringe comprising at least one component as claimed in claim 1.

14. The syringe as claimed in claim 13, comprising two components, wherein an additional friction-reducing effect results from the interaction of the respective structures of the components.
